(19) United States
(12) Patent Application Publication
(10) Pub. No.: US 2016/0008630 A1
(43) Pub. Date: Jan. 14, 2016

(54) APPARATUS FOR DETERMINING A NUMBER OF BEAMS IN IMRT

(71) Applicant: KONINKLIJKE PHILIPS N.V., AE Eindhoven (NL)

(72) Inventors: Vaitheeswaran RANGANATHAN, Bangalore (IN); Uma Satya RANJAN, Bangalore (IN)

(21) Appl. No.: 14/763,539

(22) PCT Filed: Feb. 3, 2014
(86) PCT No.: PCT/IB2014/058748

§ 371 Date: Jul. 27, 2015

Related U.S. Application Data
(60) Provisional application No. 61/760,788, filed on Feb. 5, 2013.

(51) Int. Cl. A61N 5/10

(52) U.S. Cl. CPC A61N 5/1036; A61N 5/1077; A61N 5/1045

(57) ABSTRACT

The invention relates to an apparatus for determining a number of beams in IMRT. An objective function providing unit (13) provides an objective function of an IMRT procedure, wherein the objective function depends on a target dose to be applied during the IMRT procedure. A sensitivity determination unit (14) determines the sensitivity of the objective function with respect to changes of the target dose, and a beam number determination unit (15) determines the number of beams depending on the determined sensitivity. It has been found that the sensitivity of the objective function with respect to changes of the target dose is an accurate measure for reliably determining an optimized number of beams in IMRT, which allows for generating a high quality treatment plan and which in turn leads to an improved IMRT procedure.

### FIELD OF THE INVENTION

The invention relates to an apparatus, a method and a computer program for determining a number of beams in intensity-modulated radiation therapy (IMRT). The invention relates further to an IMRT system, an IMRT method and an IMRT computer program for performing the IMRT.

### BACKGROUND OF THE INVENTION

During an IMRT procedure a planned target volume (PTV) within a person is irradiated with different beams from different directions, wherein the intensity of a beam is spatially modulated by using a multileaf collimator (MLC). Before the IMRT is applied to the person, a treatment plan is calculated depending on, for instance, the position and the shape of the PTV, the number of beams, the technical characteristics of the MLC, a prescribed target radiation dose to be applied to the PTV, the position and shape of organs at risk (OARs), et cetera. The treatment plan should be determined such that the PTV receives the prescribed target radiation dose, while the radiation dose applied to the OARs is smaller than a predefined threshold value. The quality of the treatment plan depends on inter alia the number of beams input in a respective treatment plan determination algorithm.

The article “Number and orientations of beams in intensity-modulated radiation treatments” by J. Stein et al., Medical Physics, volume 24, pages 149-160 (1997) discloses an approximate solution for determining the number of beams, which includes studying the effect of the number of beams on the quality of the treatment plan. However, only a rough estimate of the number of beams is provided, which may still lead to a non-optimal treatment plan.

### SUMMARY OF THE INVENTION

The invention relates to an apparatus, a method and a computer program for determining a number of beams in IMRT, which allow for a determination of an optimized number of beams that can be used for determining a treatment plan having an improved quality. It is a further object of the present invention to provide an IMRT system, an IMRT method and an IMRT computer program, which can be used for applying the IMRT based on the treatment plan with the improved quality.

In a first aspect of the present invention an apparatus for determining a number of beams in an IMRT procedure is presented, wherein the apparatus comprises:

an objective function providing unit for providing an objective function of the IMRT procedure, wherein the objective function depends on a target dose to be applied during the IMRT procedure,

a sensitivity determination unit for determining the sensitivity of the objective function with respect to changes of the target dose,

a beam number determination unit for determining the number of beams depending on the determined sensitivity.

It has been found that the sensitivity of the objective function with respect to changes of the target dose is an accurate measure for reliably determining an optimized number of beams in IMRT. Thus, since the sensitivity determination unit determines a sensitivity of the objective function with respect to changes of the target dose and since the beam number determination unit determines the number of beams depending on the determined sensitivity, the number of beams can be determined in a way such that, if this number of beams is used for generating a treatment plan, a high quality treatment plan can be generated, which in turn leads to an improved IMRT applied to a living object, i.e. a person or an animal, if the IMRT is applied in accordance with the treatment plan.

In an embodiment the beam number determination unit is adapted to determine a larger number of beams, if the sensitivity is larger, and a smaller number of beams, if the sensitivity is smaller. In particular, the beam number determination unit is adapted to determine the number of beams based on a dependence defining that the number of beams is proportional to the square root of the sensitivity. Determining the number of beams in this way leads to a further improved quality of the treatment plan, if the treatment plan is determined based on this optimized number of beams determined in this way.

In a preferred embodiment the objective function providing unit is adapted to provide the objective function for a predefined reference IMRT configuration. The reference IMRT configuration is preferentially predefined by a reference beam number and a reference beam distribution. In a preferred embodiment, the reference beam number is seven and the reference beam distribution is an angularly equidistant beam distribution. Using an angularly equidistant beam distribution as the reference beam distribution leads to a further improved quality of the treatment plan, if the treatment plan is generated depending on inter alia the determined number of beams. Moreover, using seven reference beams allows sampling a planned target volume and objects at risk like OARs over a 360 degrees rotation in a way being sufficient for providing the objective function, i.e. using more reference beams increases the computational time for determining the final number of beams, without substantially increasing the quality of the treatment plan generated depending on inter alia this determined number of beams.

It is preferred that the sensitivity determination unit is adapted to determine a first objective function value for a first target dose, a second objective function value for a second target dose, and the sensitivity depending on a difference between the first objective function value and the second objective function value. The target doses can be defined by using a minimal dose volume histogram (minDVH) objective. This allows determining the sensitivity accurately in a computationally relatively simple way. Moreover, since the sensitivity is determined by using objective function values, the determination of the sensitivity intrinsically accounts for geometric factors, dosimetric factors, plan-specific parameters, beam-specific parameters, algorithm-specific factors and/or machine-specific parameters.

It is also preferred that the apparatus comprises a number of segments determination unit for determining maximal numbers of segments for the beams to be used during the IMRT, wherein a maximal number of segments (NOS) of a beam defines the maximal NOS of the beam to be used during the IMRT to be planned and wherein the number of segments determination unit is adapted to determine the maximal NOS depending on the determined number of beams.

The treatment plan should be generated such that the respective NOS, which is defined in the finally generated treatment plan, is not larger than the respective maximal NOS determined by the number of segments determination unit. It has been found that determining for each beam the maximal NOS based on the determined total number of beams can lead to a further improved quality of the treatment plan, if this beam-specific, i.e. angle-specific, NOS is input into a treatment plan determination unit, wherein a relatively low NOS is defined in the treatment plan.

In a preferred embodiment the apparatus further comprises a projection image providing unit for providing, for a beam position of a certain beam, a projection image showing a projection of a planning target volume and of at least one object at risk, wherein the IMRT is to be applied to the planning target volume and radiation applied to the at least one object at risk is to be minimized, wherein the number of segments determination unit is adapted to segment the projection of the planning target volume and of the at least one object at risk in the projection image and to determine the maximal NOS for the certain beam depending on the segmented projections of the planning target volume and of at least one object at risk. The projection image is preferentially a beam's-eye-view (BEV) image.

In a further preferred embodiment the number of segments determination unit is adapted to determine a number of regions of overlap between the segmented projection of the planning target volume and the segmented projection of the at least one object at risk and to determine the maximal NOS of the certain beam depending on the determined number of overlap regions. It is also preferred that the number of segments determination unit is adapted to classify the arrangement of the segmented projections of the planned target volume and of the at least one object at risk into a predefined arrangement class and to determine the maximal NOS of the certain beam depending on the arrangement class. In an embodiment the number of segments determination unit is adapted to classify the arrangement depending on the number of segmented projections of the planned target volume and the at least one object at risk that overlap in a region of overlap. These operations further improve the quality of the determined maximal NOS, thereby further improving the quality of the treatment plan generated depending on—inter—alia these determined maximal NOS.

In another aspect of the present invention an IMRT system for performing an IMRT procedure is presented, wherein the IMRT system comprises:

an apparatus for determining a number of beams in the IMRT as defined in claim 1,

a treatment plan determination unit for determining a treatment plan for the IMRT based on the determined number of beams, and

an IMRT application device for applying the IMRT in accordance with the determined treatment plan.

The treatment plan determination unit can further be adapted to determine the treatment plan for the IMRT based on the determined number of beams and the determined maximal NOS of the beams.

In a further aspect of the present invention a method for determining a number of beams in an IMRT procedure is presented, wherein the method comprises:

providing an objective function of the IMRT by an objective function providing unit, wherein the objective function depends on a target dose to be applied during the IMRT procedure,

determining the sensitivity of the objective function with respect to changes of the target dose by a sensitivity determination unit,

determining the number of beams depending on the determined sensitivity by a beam number determination unit.

In another aspect of the present invention an IMRT method for performing an IMRT is presented, wherein the IMRT method comprises:

determining a number of beams in the IMRT as defined in claim 12,

determining a treatment plan for the IMRT based on the determined number of beams by a treatment plan determination unit, and

applying the IMRT in accordance with the determined treatment plan by an IMRT application device.

In a further aspect of the present invention a computer program for determining a number of beams in an IMRT procedure is presented, wherein the computer program comprises program code means for causing an apparatus as defined in claim 1 to carry out the steps of the method as defined in claim 12, when the computer program is run on a computer controlling the apparatus.

In another aspect of the present invention a computer program for performing an IMRT procedure is presented, wherein the computer program comprising program code means for causing an IMRT system as defined in claim 11 to carry out the steps of the IMRT method as defined in claim 13, when the computer program is run on a computer controlling the IMRT system.

It shall be understood that the apparatus for determining a number of beams in an IMRT procedure of claim 1, the IMRT system of claim 11, the method for determining a number of beams in an IMRT procedure of claim 12, the method for performing an IMRT procedure of claim 13, the computer program for determining a number of beams in an IMRT procedure of claim 14 and the computer program for performing an IMRT procedure of claim 15 have similar and/or identical preferred embodiments, in particular, as defined in the dependent claims.

It shall be understood that a preferred embodiment of the invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily an embodiment of an IMRT system for performing an IMRT procedure,

FIG. 2 exemplarily shows an objective function value depending on a used number of beams in a pancreas case,

FIG. 3 shows a flowchart exemplarily illustrating an embodiment of an IMRT method for performing an IMRT procedure,

FIG. 4 shows exemplarily a dependence of a sensitivity of an objective function on a used number of beams,

FIG. 5 shows exemplarily a dependence of an objective function on a used number of beams for a prostate case and a head and neck case,

FIGS. 6 to 8 show different arrangements of segmented projections of a PTV and at least one OAR, and

FIG. 9 shows a flowchart exemplarily illustrating a further embodiment of an IMRT method for performing an IMRT procedure.

### DETAILED DESCRIPTION OF EMBODIMENTS

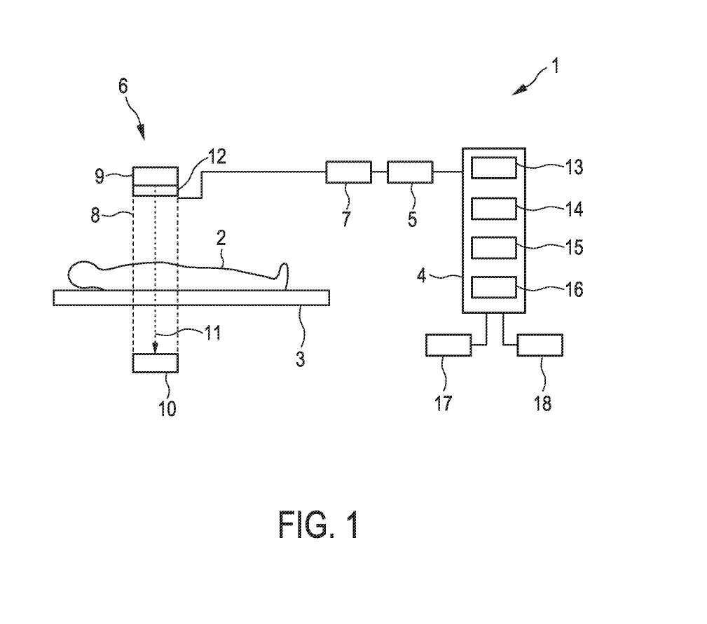

FIG. 1 shows schematically and exemplarily an embodiment of an IMRT system for performing an IMRT procedure. The IMRT system 1 comprises an apparatus 4 for determining a number of beams to be used during the IMRT procedure, a treatment plan determination unit 5 for determining a treatment plan for the IMRT procedure based on the determined number of beams and an IMRT application device 6 for applying the IMRT in accordance with the determined treatment plan.

The apparatus 4 comprises an objective function providing unit 13 for providing an objective function of the IMRT procedure, wherein the objective function depends, inter alia, on a target dose to be applied during the IMRT procedure. The apparatus 4 further comprises a sensitivity determination unit 14 for determining the sensitivity of the objective function with respect to changes of the target dose and a beam number determination unit 15 for determining the number of beams depending on the determined sensitivity.

The objective function providing unit 13 is adapted to provide the objective function for a predefined reference IMRT configuration. In this embodiment the reference IMRT configuration is predefined by a reference beam number and a reference beam distribution. Preferentially, the reference beam number is seven and the reference beam distribution is an angularly equidistant beam distribution.

The predefined IMRT configuration, which can be regarded as being a reference beam geometry condition, can be predefined by a user. The reference IMRT configuration is predefined, in order to generate reproducible results to be obtained from a sensitivity-based beam number selection (SBBNS) algorithm, which is performed by the sensitivity determination unit 14 and the beam number determination unit 15 for determining an optimal and case specific number of beams. The equispaced beam geometry of the reference IMRT configuration may be defined by following equation:

$${θ}_{i} = ( \frac{360}{N} ( i - 1 ) + {θ}_{1} ) mod 360 , \qquad ( 1 )$$

wherein θᵢ is the gantry angle of the i-th beam, N is the number of reference beams in the reference IMRT configuration and θ₁ is a constant arbitrary offset angle. In this embodiment N is seven. Seven reference beams in a plan do not prolong the dose calculation significantly and hence reduce the time needed for selecting an optimal beam number using the SBBNS algorithm. The objective function, which is provided for the reference IMRT configuration, can be described by following equation:

$$F = {\sum }_{r = 1}^{n} [ {W}_{r} f ( {D}_{r} , {V}_{r} ) ] , \qquad ( 2 )$$

wherein F is the objective function, the index r indicates the r-th objective function component (OFC), Dᵣ is the dose parameter for the r-th OFC, Vᵣ is the volume parameter of the r-th OFC component and Wᵣ is the importance factor or penalty factor of the r-th OFC component. The function f (Dᵣ,Vᵣ) represents the numerical value of the r-th OFC. The dose and volume parameters Dᵣ and Vᵣ, are defined as the clinical goals, i.e. the real objectives, of the r-th OFC. The objective function is preferentially a quadratic dose-volume based objective function. An OFC represents a certain clinical structure like a certain target element, to which the IMRT should be applied, an organ, with respect to which the applied radiation should be minimized, et cetera. The function f (Dᵣ,Vᵣ) can be a known function like the function known from the article “A gradient inverse planning algorithm with dose-volume constraints” by S. V. Spirou and C.-S. Chui, Medical Physics, volume 25, pages 321 to 333 (1998), which is herewith incorporated by reference.

After the reference IMRT configuration has been set, the SBBNS algorithm can be performed by the sensitivity determination unit 14 and the beam number determination unit 15. The SBBNS algorithm can be invoked automatically or by a user and it involves substantially two steps. In a first step, under the reference IMRT configuration, by using the provided objective function, dose-volume constrains are used as an input of the SBBNS algorithm and the sensitivity of the overall objective function score F with respect to a predefined change in a prescribed target dose is calculated. This first step is performed by the sensitivity determination unit 14. In a second step the estimated sensitivity, i.e. the calculated sensitivity, of the objective function is used to calculate the optimal beam number for the given case. This second step is performed by the beam number determination unit 15.

For determining the sensitivity in the first step a first objective function value for a first target dose and a second objective function value for a second target dose are determined, wherein the sensitivity is determined depending on a difference between the first objective function value and the second objective function value. The target doses are preferentially defined by using a minDVH objective as in the following will be described in more detail.

The minDVH objective of a PTV is preferentially used as a reference for gauging the sensitivity of the objective function. For determining the first objective function value Fₙₒᵣₘₐₗ an IMRT intensity distribution is optimized under consideration of a first target dose defined by initial parameters Dᵣ, Vᵣ and Wᵣ particularly by a corresponding first minDVH objective. The parameters used for the first objective function, in particular the first target dose, are the real clinical parameters prescribed for the actual case.

For determining the second objective function value a second IMRT intensity distribution is generated, in which the minDVH objective of the PTV is made substantially more stringent than the prescribed goal for the PTV. That means that the Dᵣ value is increased for the same volume Vᵣ, and/or for an OAR the Dᵣ value is reduced for the same volume Vᵣ, or, for the OAR, keeping the same Dᵣ value for an increased Vᵣ. For example, if the prescribed minDVH requires only 95 percent of the PTV to receive a 63 Gy dose, for calculating the second object function value F_{overconstrained} a second target dose, i.e. a second minDVH objective, can be used, which may require 100 percent of the PTV to receive 68 Gy. Based on these more stringent parameters the objective function is minimized, in order to determine the second objective function value F_{overconstrained} The sensitivity can then be calculated in accordance with following equation:

ΔF=F_{overconstrained}−Fₙₒᵣₘₐₗ. (3)

Substituting equation (2) in equation (3) leads to following equation for the sensitivity of the objective function:

$$Δ F = {\sum }_{r = 1}^{n} {[ {W}_{r} f ( {D}_{r} , {V}_{r} ) ]}_{overconstrained} - {\sum }_{r = 1}^{n} {[ {W}_{r} f ( {D}_{r} , {V}_{r} ) ]}_{normal} \qquad ( 4 )$$

In equation (4) └Wᵣf(Dᵣ,Vᵣ)┘_{overconstrained} represents the numerical value of the r-th OFC component determined for the overconstrained condition and └Wᵣf(Dᵣ,Vᵣ)┘ₙₒᵣₘₐₗ represents the numerical value of the r-th OFC under normal conditions, which correspond to the prescribed clinical parameters. In another embodiment for defining the sensitivity in equation (4) the weights Wᵣ can be omitted, because they are independent of the target dose. Essentially the sensitivity of the objective function is a measure of how much the objectives of surrounding normal structures are perturbed for any change in the PTV target dose prescription. Overconstraining the minDVH requirements results in sacrificing the sparing of other structures such that F_{overconstrained} is generally larger than Fₙₒᵣₘₐₗ.

The beam number determination unit 15 is adapted to determine a larger number of beams, if the sensitivity is larger, and a smaller number of beams, if the sensitivity is smaller. In particular, the beam number determination unit is adapted to determine the number of beams based on a dependence defining that the number of beams is proportional to the square root of the sensitivity in accordance with following equation:

Nₒₚₜᵢₘ=k·\sqrt{ΔF_{α}}, (5)

wherein Nₒₚₜᵢₘ is the optimal number of beams to be determined by the SBBNS algorithm, k is a predefined proportionality constant, which may be regarded as being a beam number constant and which can be defined in a calibration step, and ΔF_{α}represents the sensitivity of the objective function. The square root of the sensitivity of the objective function is used in this embodiment, because in this embodiment the IMRT objective function is essentially a quadratic function over dose and volume parameters. In equation (5) the notation a indicates that the calculation of the sensitivity of the objective function should be performed under the predefined reference beam geometry condition, i.e. the predefined reference IMRT configuration. The constant k is preferentially 17.65. A general calibration technique for determining the constant k will be exemplarily described in the following.

The parameter k can be calculated under the reference beam geometry condition using a reference case for which the optimal beam number is known. The constant k can then be determined in accordance with following equation.

$$k = {( \frac{{N}_{optim}^{known}}{\sqrt{Δ {F}_{α}}} )}_{β} , \qquad ( 6 )$$

wherein Nₒₚₜᵢₘ^{known} denotes the known optimal beam number for the reference case under reference beam geometry conditions. The index fl denotes the reference case, i.e. the reference patient case.

In this example the reference case is a pancreas case with tight dose-volume constrains to the PTV and other normal structures. The reference beam geometry condition, i.e. the reference IMRT configuration, involves seven equispaced reference beams as mentioned above. The following table provides a list of other reference conditions adopted in this example, while calculating the beam number constant k.

| reference conditions | values/specifications | |
|---|---|---|
| first target dose prescription | 60 | Gy |
| second target dose prescription | 65 | Gy |
| Total NOS | 60 | |
| Minimum Segment Area | 4 | cm² |
| Minimum MU | 4 | MU/segment |
| Beam Energy | 6 | MV |
| Beam Penumbra | 0.7 | cm |

| | |
|---|---|
| LINAC | Elekta Synergy |
| MLC Leakage | ≈0.5% |
| technique | DMPO |

In this table the total number of segments is the number given by a user, which defines the maximal number of segments for the treatment plan and which is used as a constraint to the conversion algorithm. The minimum segment area (MSA) is a further constraint to the conversion algorithm defining the minimum area of each resulting segment. If the conversion algorithm produces a segment being smaller than the minimum segment area, this segment will be eliminated or the segment area will be maximized by the algorithm. The minimum MU is a further constraint to the conversion algorithm defining the minimum MU for each resulting segment. If the conversion algorithm produces a segment having an MU being smaller than the minimum MU, this segment will be eliminated or the MU of this segment will be increased to the minimum MU. The abbreviation LINAC means linear particle accelerator, and the term “technique” refers to the technique used for optimization and conversion, wherein in this example a direct machine parameters optimization (DMPO) technique has been used.

The reference conditions mentioned in the previous table are set, in order to make the calculation of the beam number constant k reproducible. In order to determine the optimal beam number for the present reference case, the objective function value, i.e. the final objective function score, was calculated for different beam numbers starting from 3 beams to 15 beams. For all of these objective function minimization calculations, i.e. for all of the corresponding treatment plan calculations, the dose-volume constrains were kept the same, in order to allow for an effective comparison of the different calculations performed with the different beam numbers.

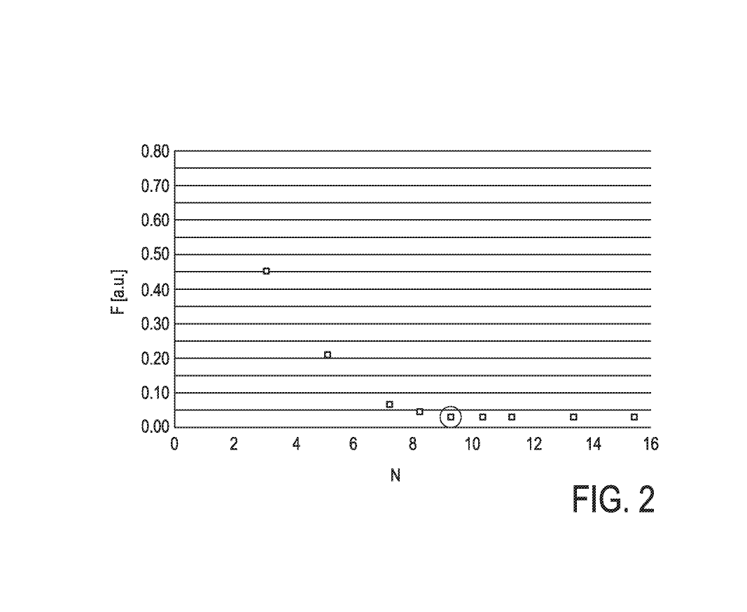

FIG. 2 shows the objective function values F in arbitrary units depending on the number of beams N. The lower the objective function value, i.e. the objective function score, the better is the quality of the treatment plan, which corresponds to the objective function value. Therefore, a beam number corresponding to a relatively low objective function value is chosen to be an optimal beam number. As can be seen in FIG. 2, at the beam number 9 the objective function value starts to saturate. This indicates that there is not going to be any considerable improvement in the dose distribution beyond 9 beams for the reference case. If more than 9 beams are used, it will result in an unnecessary increment in monitor units (MU) and beam segments, without any appreciable improvement in the dose distribution. Hence, it can be concluded that 9 beams is an optimal choice for the chosen reference case.

After in this example the known optimal beam number Nₒₚₜᵢₘ^{known} has been determined as being 9, for the reference case the sensitivity of the objective function can be determined as described above with reference to equations (2) to (4) and the resulting sensitivity together with the optimal beam number can be used to calculate the constant k in accordance with following equation:

$$k = {( \frac{{N}_{optim}^{known}}{\sqrt{Δ {F}_{α}}} )}_{β} = {( \frac{{N}_{optim}^{known}}{\sqrt{{F}_{overconstrained} - {F}_{normal}}} )}_{β} = {( \frac{9}{\sqrt{0.3235 - 0.063}} )}_{β} = {( \frac{9}{0.51} )}_{β} \approx 17.65 \qquad ( 7 )$$

Generally, the beam number constant value 17.65 may be changed, if a value of the reference conditions shown in the above mentioned table is modified. However, a huge change in k is not expected, because the numerator of the beam number constant term shown in equation (6) will change correspondingly to any change in the denominator term occurring due to alterations in the reference conditions. Thus, the beam number constant k=17.65 may be valid for a lot of different cases.

Since the SBBNS algorithm uses the sensitivity of the objective function to calculate the optimal beam number, it intrinsically accounts for various influencing factors like geometric factors, dosimetric factors, plan-specific parameters, beam-specific parameters, algorithm-specific factors and machine-specific parameters. The geometric factors are, for instance, the sizes and shapes of anatomical structures, their overlaps, et cetera. These geometric factors can be provided by a segmented three-dimensional image of a person 2 lying on a support element 3 like a patient table, to which the IMRT procedure is to be applied. The segmented three-dimensional image shows, for instance, the PTV and OARs.

Dosimetric factors are, for instance, dose-volume constraints, penalties, et cetera. Plan-specific parameters are, for instance, the NOS, the minimum segment size, the minimum MU, et cetera. Beam-specific parameters may be, for example, the beam energy, the beam penumbra, et cetera. Moreover, algorithm-specific factors can be the kind of optimization algorithm, the kind of dose calculation algorithm, et cetera. And, machine-specific parameters can be, for example, mechanical and/or dosimetric properties of the MLC.

Based on the determined optimal beam number the treatment plan determination unit 5 can determine the treatment plan for the IMRT therapy by using known fluence optimization and conversion algorithms like the algorithm disclosed in the article “Multileaf collimator leaf sequencing algorithm for intensity modulated beams with multiple static segments” by P. Xia and L. J. Verhey, Medical Physics, volume 25, pages 1424 to 1434 (1998).

The SBBNS algorithm cannot only be applied in a pancreas case, but also in other cases like in head and neck cases, prostate cases, lung cases, et cetera.

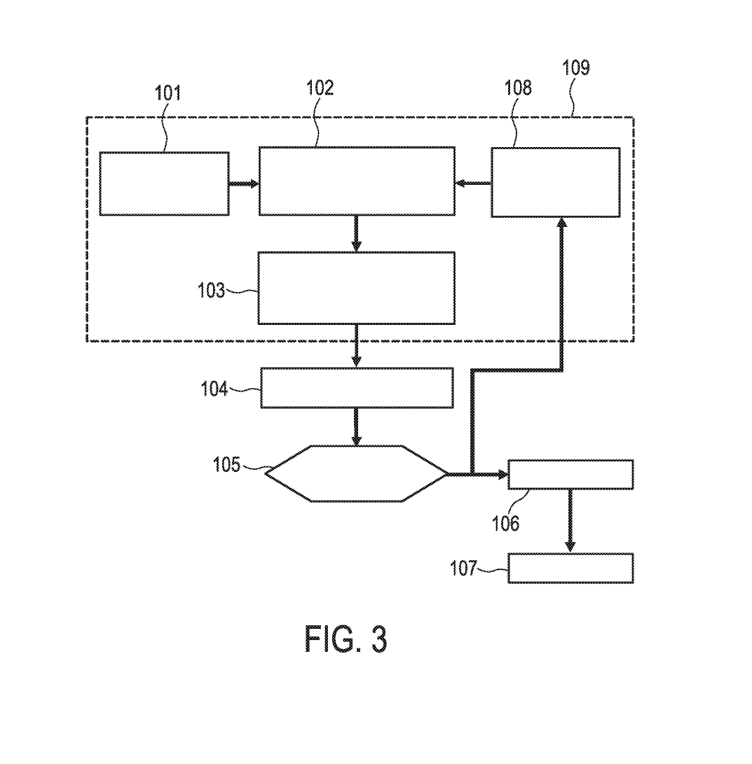

In the following an embodiment of an IMRT method for performing an IMRT procedure will exemplarily be described with reference to a flowchart shown in FIG. 3.

In step 101 a reference beam geometry condition, i.e. a reference IMRT configuration, is created, which comprises seven equispaced beams, and a corresponding objective function is provided, which depends on a target dose to be applied during the IMRT procedure. In step 102 the sensitivity of the objective function with respect to changes of the target dose is determined, and in step 103 the optimal number of beams is determined depending on the determined sensitivity of the objective function. In step 104 a fluence optimization and conversion algorithm is performed based on the determined optimal number of beams, in order to generate a treatment plan. In step 105 it is determined whether the dose distribution, which will result from the treatment plan, is acceptable. If this is the case, the treatment plan is output in step 106 and in step 107 the IMRT procedure is carried out in accordance with the treatment plant. If the dose distribution resulting from the treatment plan is not acceptable, in step 108 dose-volume constraints and penalties, i.e. Dᵣ, Vᵣ and Wᵣ, are defined, in particular redefined, in step 108 and steps 102 to 104 are performed again based on the defined or redefined, respectively, dose-volume constraints and penalties. Steps 102 to 105 and 108 are performed, until a treatment plan providing an acceptable dose distribution has been determined.

The dashed box 109 indicates in FIG. 3 the SBBNS algorithm. Thus, the SBBNS algorithm can comprise steps 101 to 103 and 108. However, the SBBNS algorithm can also be regarded as comprising only steps 101 to 103, which are sufficient for determining the beam number Nₒₚₜᵢₘ.

Steps 101 to 103 can be regarded as being steps of a method for determining a number of beams in an IMRT procedure.

IMRT procedures may be broadly classified into: (1) static IMRT (sIMRT) and (2) rotational IMRT (rIMRT). In static IMRT (either segment-based or dynamic IMRT), the fundamental factors that determine the quality of a plan are the number of beams and their angles. There are a number of beam angle optimization (BAO) algorithms in the literature, which give case specific solution to the beam angle problem. Many radiotherapy departments have gradually stared implementing BAO algorithms into routine clinical practice. However, prior to the beam angle optimization, many of the BAO algorithms require the input of number of beams to be used for a given plan. In the current practice, the beam numbers are decided either through the experience of the planner or by a trial-and-error process, which may not guarantee a suitable beam number in many clinical situations. Adding more beams in static IMRT beyond a point merely increases the MUs and NOS without any considerable improvement in dose distribution, leading to more leakage radiation and increased critical organ dose. Producing a good dose distribution with lesser number of MUs and segments fundamentally requires an appropriate selection of number of beams and their angles.

The apparatus 4 provides therefore a case specific beam number for an IMRT procedure, which can be determined very fast, in order to encourage the user to frequently use it in routine practice. The apparatus 4 provides a good balance between superior dose distribution and excessive complexity, which can be easily implemented. It is preferentially used in segment-based IMRT.

A user generally does not have a systematic guidance to choose the number of beams in IMRT, thereby increasing inter-user variability in the delivery plan, i.e. the treatment plan, quality. Moreover, a manual specification of the number of beams, which is far-off from the optimal value, would rigorously affect the quality and ability of a treatment plan. And, in complex clinical situations, it is generally not easy to determine the total number of beams without many trial-and-error steps. Furthermore, a manual selection of the number of beams is time consuming for complex clinical situations. The apparatus 4 overcomes these drawbacks by using the SBBNS algorithm, which fast and accurately determines an optimal beam number for an IMRT procedure.

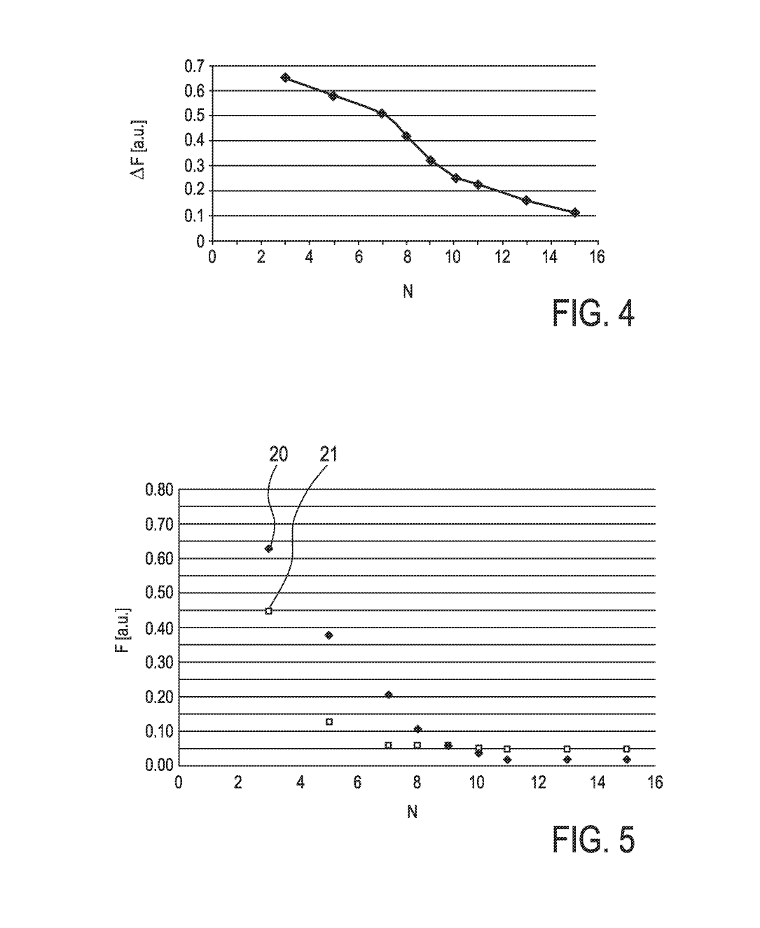

The objective function is an overall estimate of the treatment plan quality, in particular in single-criteria optimization. The smaller the value of the objective function, the better is the quality of the dose distribution in IMRT. The SBBNS algorithm is based on the behaviour that the sensitivity of the objective function to the prescribed target dose is inversely proportional to the total number of beams used in a given case. This is exemplarily shown in FIG. 4, in which the objective function sensitivity ΔF is displayed in arbitrary units with respect to the used number of beams N. In the example shown in FIG. 4 the sensitivity of the objective function is determined with respect to a 5 Gy increment in the prescribed target dose for a pancreas case. If a smaller number of beams is used, the objective function is more sensitive to any change in the target dose. Similarly, if a larger number of beams is used, the objective function is less sensitive to any change in the target dose. In other words, a lower level of sensitivity of the objective function indicates that there are enough beams present for ensuring the normal tissue sparing, if the prescribed target dose is increased. Likewise, if the objective function is too sensitive to the target dose, it indicates that there is not a large enough number of beams present for ensuring the normal organ sparing, when the prescribed target dose is increased. Stated differently, if the objective function sensitivity is found to be very high under reference beam geometry condition, the number of beams required to produce an optimal dose distribution is larger, in order to reduce the sensitivity to a sufficient level. Therefore, the required number of beams for a given case is larger, if the sensitivity of the objective function obtained under reference beam geometry condition is larger. For instance, the required number of beams can be proportional to the sensitivity of the objective function or to the square root of the sensitivity of the objective function as described above with reference to equation (5). The SBBNS algorithm exploits this behaviour for determining the optimal beam number for the respective case.

FIG. 5 illustrates the dependence of the objective function F on the used number of beams N for a first case being a prostate case and a second case being a head and neck case. In FIG. 5 the squares 21 indicate the results of the prostate case and the diamonds 20 indicate the results of the head and neck case. As can be seen in FIG. 5, the saturation of the objective function value, i.e. of the final objective function score, starts at beam number 7 for the prostate case and at beam number 11 for the head and neck case. Thus, the optimal number of beams, which will be determined by the SBBNS algorithm, will be 7 for the prostate case and 11 for the head and neck case.

Referring again to FIG. 1, the IMRT application device 6 comprises a rotatable gantry 8, on which a radiation source 9 and a radiation detector 10 are mounted. The radiation source 9 is adapted to emit a beam 11 for performing the IMRT in accordance with the treatment plan. Before carrying out the IMRT in accordance with the treatment plan, a BEV image may be generated by using the radiation detector 10, wherein in this case the intensity of the beam 11 is relatively small, because it is just used for generating the BEV image. The BEV image may be shown on a display 18 of the system 1. In front of the radiation source 9 a MLC 12 is present, in order to allow the IMRT application device 6 to modify the beam 11 in accordance with the treatment plan. The IMRT application device 6 further comprises a control unit 7 for controlling the gantry with the radiation source, the radiation detector and the MLC such that the IMRT is carried out in accordance with the treatment plan. The system 1 further comprises an input unit 17 like a keyboard, a mouse, a touchpad, et cetera for allowing a user to provide inputs to the system, for instance, to initiate the SBBNS algorithm or to initiate other operations to be performed by the system 1.

The apparatus 4 can further comprise a number of segments determination unit 16 for determining maximal number of segments for the beams to be used during the IMRT procedure, wherein the number of segments determination unit 16 is adapted to determine the maximal NOS depending on the determined number of beams. Preferentially, the apparatus 4 further comprises a projection image providing unit for providing, for a beam position of a certain beam, a projection image showing a projection of a PTV and of at least one OAR, wherein the IMRT is to be applied to the PTV and radiation applied to the at least one OAR is to be minimized, wherein the number of segments determination unit 16 is adapted to segment the projection of the PTV and of the at least one OAR in the projection image and to determine the maximal NOS for the certain beam depending on the segmented projections of the PTV and the at least one OAR. In this embodiment, the projection image is a BEV image generated by the radiation source 9 and the radiation detector 10 for the respective beam such that the projection image providing unit is the IMRT application device 6. However, in another embodiment the projection image providing unit can also be another imaging unit or an projection image receiving unit for receiving the projection image from the IMRT application device 6 or from another imaging unit and for providing the received projection image.

Since the number of segments determination unit 16 is adapted to determine the maximal NOS for a certain beam depending on the segmented projections of the PTV and of the at least one OAR being indicative of an anatomy within the person 2, the algorithm for determining the maximal NOS for a certain beam can be regarded as being an anatomy-guided segment counting (AGSC) algorithm that arrives at an optimal maximal NOS per beam.

The number of segments determination unit 16 is preferentially adapted to determine a number of regions of overlap between the segmented projection of the PTV and the segmented projection of the at least one OAR and to determine the maximal NOS of the certain beam depending on the determined number of overlap regions. The number of segments determination unit 16 is preferentially further adapted to classify the arrangement of the segmented projections of the PTV and of the at least one OAR into a predefined arrangement class and to determine the maximal NOS of the certain beam depending on the arrangement class, wherein the arrangement is classified into an arrangement class depending on the number of segmented projections of the PTV and the at least one OAR that overlap in a region of overlap.

In particular, the AGSC algorithm preferentially takes as input the anatomical projections in the respective BEV image, in order to determine the areas of overlap, wherein the number of regions overlapping with the PTV is counted and a combinatorial combination of these regions is computed. This can be computed from a library of all possible Boolean combinations applicable to a set of structures. The computed combination of sub regions along with other planning parameters such as, but not restricted to, the minimum segment size (MSS), the region-of-interest (ROI) importance weighting factor, et cetera can be used by the AGSC algorithm to arrive at the maximum NOS per beam. This will in the following be described in more detail with reference to FIGS. 7 to 9.

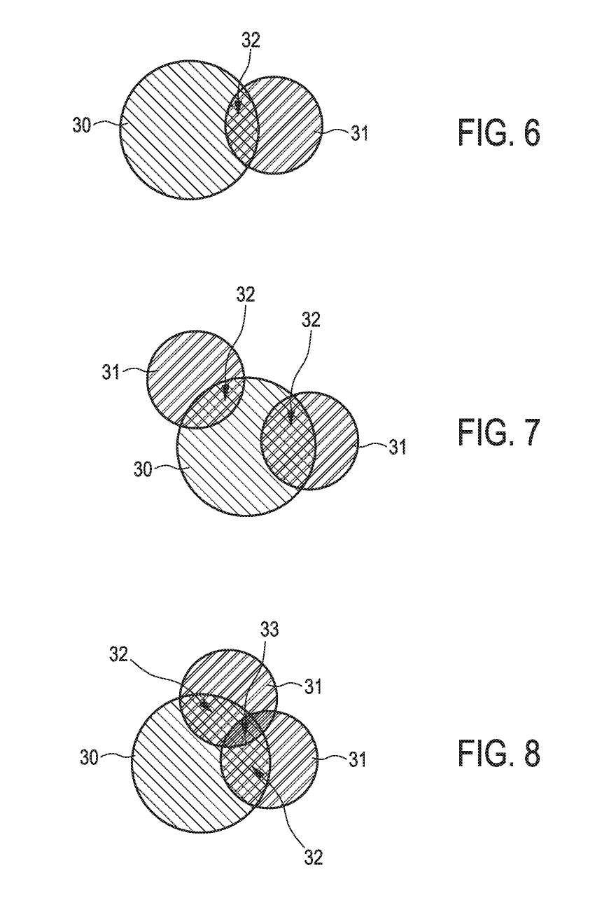

FIG. 6 shows an arrangement of a segmented projection 30 of a PTV and a segmented projection 31 of an OAR, which comprise a single overlapping region 32. Such an arrangement of the segmented projections having a single overlapping region can be assigned to a first arrangement class. FIG. 7 shows a further arrangement of a segmented projection 30 of a PTV and of segmented projections 31 of two OARs. This arrangement comprises two overlapping regions 32. Such an arrangement having two overlapping regions 32 can be assigned to a second arrangement class. FIG. 8 shows an arrangement with one segmented projection 30 of a PTV and two segmented projections 31 of an OAR, which comprises two overlapping regions 32 with a single overlap, i.e. where only two objects overlap, and one overlapping region 33 with an overlap of more than two objects. The arrangement shown in FIG. 8 can be assigned to a third arrangement class.

For each arrangement of the segmented projections of the PTV and of the at least one OAR a combinatorial term can be determined, which describes the different overlapping and non-overlapping regions of the segmented projection of the PTV. For instance, for the arrangement shown in FIG. 6 following combinatorial term can be defined:

1. T,

2. T∩O1,

3. T−{T∩O1} (8)

wherein T indicates the segmented projection 30 of the PTV and O1 indicates the segmented projection 31 of the OAR shown in FIG. 6. The combinatorial term for the arrangement shown in FIG. 7 can be defined by:

1. T,

2. T∩O1,

3. T−{T ∩O1},

4. T∩O2,

5. T−{T∩O2},

6. T−{T∩O1}−{T∩O2} (9)

wherein the segmented projections 31 of the OARs are represented by O1 and O2, respectively. For the arrangement shown in FIG. 8 the combinatorial term can be calculated as follows:

1. T,

2. T∩O1,

3. T−{T ∩O1},

4. T∩O2,

5. T−{T∩O2},

6. T−{T∩O1}−{T∩O2},

7. {T∩O1}+{T∩O2},

8. T−{(T∩O1)∩(T∩O2)},

9. {T∩O2}∩{T∩O2} (10)

The ASC algorithm can use predefined assignments between maximal NOS and all possible combinatorial terms. However, it is preferred that the respective arrangement is assigned to a predefined arrangement class, wherein the respective maximal NOS is determined based on the respective arrangement class, the number of overlap regions and optionally further parameters like the MSS. For instance, all arrangements with only a single OAR, which may or may not form an overlapping region with the PTV in the BEV image, can be assigned to the first arrangement class. All arrangements having more than one OAR, wherein each OAR forms a single overlap region with the PTV in the respective BEV image, can be assigned to the second arrangement class. For instance, the arrangement shown in FIG. 7 can be assigned to the second arrangement class. Moreover, all arrangements having more than one OAR in the respective BEV image, wherein all OARs form a multiple overlap region with a respective at least one other OAR and the PTV in the respective BEV image, can be assigned to the third arrangement class. For instance, as already mentioned above, the arrangement shown in FIG. 8 can be assigned to this third arrangement class. If an arrangement comprises several OARs in a BEV image, wherein the arrangement comprises at least one single overlapping region 32 as exemplarily shown in FIG. 7 and at least one multiple overlap region 33 as exemplarily shown in FIG. 8, this arrangement may be assigned to a fourth arrangement class. In the following a table is shown, which may be used by the number of segments determination unit 16 for determining the respective maximal NOS per beam depending on the actual arrangement class and the number of overlapping regions in the actual arrangement.

| number of | maximum NOS per beam | | |
|---|---|---|---|
| OAR overlaps | first class | second class | third class |
| 0 | 1 | nil | nil |
| 1 | 3 | nil | nil |
| 2 | nil | 6 | 9 |
| 3 | nil | 9 | 14 |
| 4 | nil | 12 | 19 |
| 5 | nil | 15 | 24 |

If the size of an overlap region, i.e. the overlap size of an overlap region, is smaller than a predefined MMS, which may be predefined by a user like a physician, the respective overlapping region is preferentially not counted, when determining the number of overlapping regions in the respective arrangement.

Based on the given BEV image, the AGSC algorithm preferentially automatically chooses the respective type of ROI overlap, i.e. the respective arrangement class, and counts the overlapping regions, wherein based on the chosen arrangement class and the number of overlapping regions the maximal NOS per beam can be determined by using, for instance, the previously shown table.

The treatment plan determination unit 5 can be adapted to determine the treatment plan for the IMRT procedure based on the determined total number of beams and based on the determined maximal NOS determined for each respective beam. Thus, these numbers can be fed into the above mentioned algorithm for fluence optimization and conversion. The algorithm uses the constraints of the total number of beams and the angle-specific maximal NOS, i.e. the maximal NOS determined for the respective beam, and determines the final deliverable beam segments and fluence and thus the treatment plan accordingly. The IMRT application device 6 can then apply the IMRT in accordance with the determined treatment plan.

In an embodiment, the determined maximal NOS per beam can be displayed to the user by using the display 18, in order to allow the user to check whether he/she agrees with the determined maximal NOS per beam and in order to allow him/her to modify the respective maximal NOS per beam, if desired. The treatment plan can then be determined based on the optionally modified maximal NOS per beam.

The maximal NOS per beam can be used as a constraint by the fluence optimization and conversion algorithm, which determines the treatment plan. For instance, the fluence optimization and conversion algorithm disclosed in the article “Direct aperture optimization: A turnkey solution for step-and-shoot IMRT” by D. M. Shepard, M. A. Earl, X. A. Li, S. Naqvi, and C. Yu, Medical Physics, volume 29, number 6 (2002), which uses the maximal NOS as a constrained and which is herewith incorporated by reference, can be used. However, also other known fluence optimization and conversion algorithms can be used, which use the maximal NOS as a constraint.

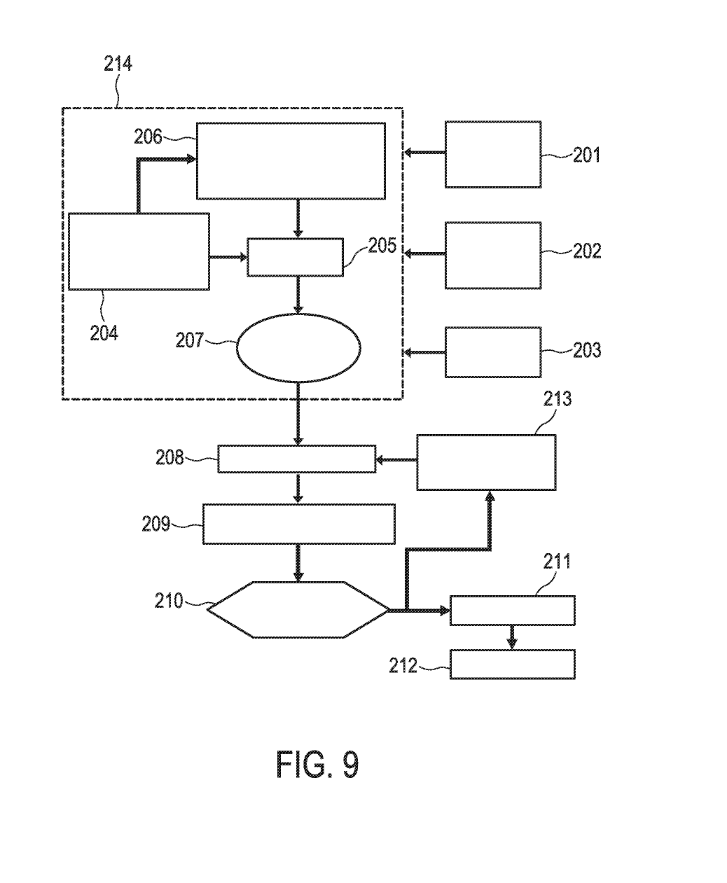

In the following a further embodiment of an IMRT method for performing an IMRT procedure will exemplarily be described with reference to a flowchart shown in FIG. 9.

In step 201 the number of beams determined by the beam number determination unit 15 is provided to the AGSC algorithm 214 performed by the number of segments determination unit 16. Moreover, in step 201 a user can input the angular positions of the beams via the input unit 17. Alternatively, the angular positions can be predefined as being, for instance, angularly equidistant. In step 202, optionally ROI importance weighting factors, i.e. parameters W are input into the algorithm 214 via the input unit 17 by the user.

In step 203 optionally further planning parameters are fed into the AGSC algorithm such as an overlap penalty. The user may wish to penalize certain types of overlaps such that the number of segments seeing a particular overlap region can be controlled to an extent. This can be done by inputting the overlap penalty into the AGSC algorithm 214. In step 204 a user defined MSS value is provided, which is used together with other parameters in step 206, in order to construct a Boolean table, which provides assignments between the number of overlap regions and arrangement classes to the maximal NOS of the respective beam. The Boolean table can be constructed in accordance with the technique disclosed in, for instance, the article “An optimized forward planning technique for intensity modulated radiation therapy” by Y. Xiao, J. Galvin, M. Hossain, and R. Valicenti, Medical Physics, volume 27, pages 2093 to 2099 (2000), which is herewith incorporated by reference.

In step 205 for each beam, i.e. for each angular position, segmented projections of the PTV and at least one OAR are determined in a BEV image, wherein based on these segmented projections the arrangement class and the number of overlapping regions is determined, which are used together with the Boolean table for determining the angle-specific maximal NOS 207. In step 208 the treatment plan determination unit 5 performs a fluence optimization algorithm for meeting clinical objectives input into the system by the user via the input unit 17. For performing this fluence optimization known fluence optimization algorithms can be used like the fluence optimization algorithm disclosed in the article “Algorithm and functionality of an intensity modulated radiotherapy optimization system” by Q. Wu and R. Mohan, Medical Physics, volume 27, pages 701 to 711 (2000), which is herewith incorporated by reference.

In step 209 the treatment plan determination unit 5 invokes a conversion algorithm for obtaining deliverable segments under consideration of the angle-specific maximal NOS received from the AGSC algorithm 214. For performing this conversion algorithm a known algorithm can be used like the conversion algorithm disclosed in the article “Multileaf collimator leaf sequencing algorithm for intensity modulated beams with multiple static segments” by P. Xia and L. J. Verhey, Medical Physics, volume 25, pages 1424 to 1434 (1998), which is herewith incorporated by reference.

In step 210 it is checked whether the dose distribution, which would result from the treatment plan determined in step 209, would be acceptable. If this is the case, in step 211 the treatment plan is provided to the IMRT application device 6, whereupon the treatment plan is executed by the IMRT application device 6 in step 212.

In particular, in step 210 it is checked whether the dose distribution indicates that the PTV receives the prescribed target dose and whether the dose applied to the one or several OARs is smaller than predefined dose limits. In step 210 further parameters may be considered for determining whether the dose distribution is acceptable like the conformity index, which provides an estimation of how well the prescribed target dose is confined within the PTV, and the homogeneity index, which provides an indication of how well the prescribed target dose is homogeneously spread inside the PTV.

If it has been determined in step 210 that the dose distribution is not acceptable, in step 213 dose-volume constraints and penalties are defined, in particular redefined, and the method continues with step 208. Steps 208 to 210 and 213 are performed, until the dose distribution is determined as being acceptable in step 210.

In inverse planning for IMRT/direct machine parameters optimization (DMPO), conversion of the fluence profile to a deliverable beam configuration is an important step. The conversion is performed by a variety of algorithms based on either clustering methods or optimization methods as in DMPO. The output of the conversion algorithm is a set of MLC segments, i.e. beam segments, and their associated MUs. For accurate delivery, an optimal NOS with reasonable MUs is essential. Multiple small MU segments, i.e. beam segments, are not robust to an accelerator's performance and take a longer time to deliver, thereby increasing the probability of patient motion induced errors during delivery. Too few segments are not conformal to the dose. In most planning systems, dose conformity is well measured and the problem of too few segments does not arise. However, oversegmentation may be a problem, especially in complex anatomical structures such as head and neck or a small tumor area, for instance gross tumor volume (GTV), within a bigger tumor volume, which might be the PTV, thereby needing an integrated boost. This typically leads to a 40 percent increase in NOS over a case which does not need an additional simultaneous boost. This results in a disproportionately large NOS in a small area.

In order to increase the deliverability of a plan, a post-processing approach may be used, wherein the segments are post-processed using methods such as smoothing for reducing the NOS and thereby increasing the delivery efficiency. However, clinically, such post-processing can produce unforeseen degradation in the plan quality and cause reduced target coverage.

It is therefore preferred to solve the underlying problem by incorporating the maximum NOS directly into the optimization process, in order to provide a good initialization condition for the conversion/DMPO algorithm by specifying a near-optimal NOS for each beam depending on the anatomical configuration as obtainable from the BEV.

The number of segments determination unit 16 and the corresponding AGSC algorithm can overcome following disadvantages of known IMRT systems. For instance, current systems do not provide a systematic guidance for choosing the maximal NOS, thereby increasing the inter-user variability with respect to the delivery plan, i.e. the treatment plan, quality. Moreover, if the maximal NOS is manually specified, a non-optimal value can be input into the treatment plan generation algorithm, which can rigorously affect the performance of the treatment plan determination algorithm, i.e. of the optimizer. Moreover, in known systems in case of a complex anatomical layout such as head and neck structures and cases of a small tumor area requiring a simultaneous integrated boost the NOS typically increases by 40 percent over a small area. This affects the dose linearity in the tumor region, which can lead to an inaccurate dose computation and delivery.

Variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Operations like the provision of the objective function, the determination of the sensitivity of the objective function, the determination of the number of beams, the determination of the NOS per beam or per angular position of the beam, et cetera performed by one or several units or devices can be performed by any other number of units or devices. For example, steps 101 to 105, 108, 206 to 210 and 213 can be performed by a single unit or by any other number of different units. The operations and/or the control of the IMRT system in accordance with the IMRT method and/or the control of the apparatus for determining the number of beams in IMRT in accordance with the method for determining a number of beams in IMRT can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

The invention relates to an apparatus for determining a number of beams in IMRT. An objective function providing unit provides an objective function of an IMRT procedure, wherein the objective function depends on a target dose to be applied during the IMRT procedure. A sensitivity determination unit determines the sensitivity of the objective function with respect to changes of the target dose, and a beam number determination unit determines the number of beams depending on the determined sensitivity. It has been found that the sensitivity of the objective function with respect to changes of the target dose is an accurate measure for reliably determining an optimized number of beams in IMRT, which allows for generating a high quality treatment plan and which in turn leads to an improved IMRT procedure.

1. An apparatus for determining a number of beams in intensity-modulated radiation therapy, the apparatus comprising:
an objective function providing unit for providing an objective function of the intensity-modulated radiation therapy, wherein the objective function depends on a target dose to be applied during the intensity-modulated radiation therapy,
a sensitivity determination unit for determining the sensitivity of the objective function with respect to changes of the target dose,
a beam number determination unit for determining the number of beams depending on the determined sensitivity.

2. The apparatus as defined in claim 1, wherein the beam number determination unit adapted to determine a larger number of beams, if the sensitivity is larger, and a smaller number of beams, if the sensitivity is smaller.

3. The apparatus as defined in claim 2, wherein the beam number determination unit is adapted to determine the number of beams based on a dependence defining that the number of beams is proportional to the square root of the sensitivity.

4. The apparatus as defined in claim 1, wherein the objective function providing unit is adapted to provide the objective function for a predefined reference intensity-modulated radiation therapy configuration.

5. The apparatus as defined in claim 1, wherein the sensitivity determination unit is adapted to determine
a first objective function value for a first target dose,
second objective function value for a second target dose, and
the sensitivity depending on a difference between the first objective function value and the second objective function value.

6. The apparatus as defined in claim 1, wherein the apparatus further comprises a number of segments determination unit for determining maximal numbers of segments for the beams to be used during the intensity-modulated radiation therapy, wherein the number of segments determination unit is adapted to determine the maximal number of segments depending on the determined number of beams.

7. The apparatus as defined in claim 6, wherein the apparatus further comprises a projection image providing unit for providing, for a beam position of a certain beam, a projection image showing a projection of a planning target volume and of at least one object at risk, wherein the intensity-modulated radiation therapy is to be applied to the planning target volume and radiation applied to the at least one object at risk is to be minimized, wherein the number of segments determination unit is adapted to segment the projection of the planning target volume and of the at least one object at risk in the projection image and to determine the maximal number of segments for the certain beam depending on the segmented projections of the planning target volume and of at least one object at risk.

8. The apparatus as defined in claim 7, wherein the number of segments determination unit is adapted to determine a number of regions of overlap between the segmented projection of the planning target volume and the segmented projection of the at least one object at risk and to determine the maximal number of segments of the certain beam depending on the determined number of overlap regions.

9. The apparatus as defined in claim 7, wherein the number of segments determination unit adapted to classify the arrangement of the segmented projections of the planned target volume and of the at least one object at risk into a predefined arrangement class and to determine the maximal number of segments of the certain beam depending on the arrangement class.

10. The apparatus as defined in claim 9, wherein the number of segments determination unit is adapted to classify the arrangement depending on the number of segmented projections of the planned target volume and the at least one object at risk that overlap in a region of overlap.

11. An intensity-modulated radiation therapy system for performing an intensity-modulated radiation therapy, the intensity-modulated radiation therapy system comprising:
an apparatus for determining a number of beams in the intensity-modulated radiation therapy as defined in claim 1,
a treatment plan determination unit for determining a treatment plan for the intensity-modulated radiation therapy based on the determined number of beams, and
an intensity-modulated radiation therapy application device for applying the intensity-modulated radiation therapy in accordance with the determined treatment plan.

12. A method for determining a number of beams in intensity-modulated radiation therapy, the method comprising:
providing an objective function of the intensity-modulated radiation therapy by an objective function providing unit, wherein the objective function depends on a target dose to be applied during the intensity-modulated radiation therapy,
determining the sensitivity of the objective function with respect to changes of the target dose by a sensitivity determination unit,
determining the number of beams depending on the determined sensitivity by a beam number determination unit.

13. An intensity-modulated radiation therapy method for performing an intensity-modulated radiation therapy, the intensity-modulated radiation therapy method comprising:
determining a number of beams in the intensity-modulated radiation therapy as defined in claim 12,
determining a treatment plan for the intensity-modulated radiation therapy based on the determined number of beams by a treatment plan determination unit, and
applying the intensity-modulated radiation therapy in accordance with the determined treatment plan by an intensity-modulated radiation therapy application device.

14. A computer program for determining a number of beams in intensity-modulated radiation therapy, the computer program comprising program code means for causing an apparatus to carry out the steps of the method as defined in claim 12, when the computer program is run on a computer controlling the apparatus.

15. A computer program for performing an intensity-modulated radiation therapy, the computer program comprising program code means for causing an intensity-modulated radiation therapy system to carry out the steps of the intensity-modulated radiation therapy method as defined in claim 13, when the computer program is run on a computer controlling the intensity-modulated radiation therapy system.
